(19) United States
(12) Patent Application Publication
(10) Pub. No.: US 2008/0114468 A1
(43) Pub. Date: May 15, 2008

(54) Processes for making ceramic medical devices

(75) Inventor: Mukesh Kumar, Warsaw, IN (US)

Correspondence Address: HARNESS, DICKEY & PIERCE, P.L.C., P.O. BOX 828, BLOOMFIELD HILLS, MI, 48303, US

(73) Assignee: Biomet Manufacturing Corp., Warsaw, IN (US)

(21) Appl. No.: 11/595,134

(22) Filed: Nov. 10, 2006

(51) Int. Cl. A61F 2/28; C04B 35/01; H05B 6/80

(52) U.S. Cl. 623/23.56; 501/97.3; 264/434

(57) ABSTRACT

A process for making a sintered ceramic medical device, comprising providing an unsintered ceramic composition, forming the unsintered ceramic composition into a green body that comprises unsintered ceramic, irradiating the green body with microwave radiation, and cooling the sintered body. The microwave radiation has a frequency capable of heating the unsintered ceramic to a temperature sufficient to sinter the green body, thereby preparing a sintered ceramic medical device. A medical device comprising volumetrically sintered ceramic, and a volumetrically sintered ceramic are also disclosed.

### BACKGROUND

The present disclosure relates generally to processes for making volumetrically sintered ceramic medical devices.

Many ceramic (or ceramic composite) materials are used in the production of medical devices. For example, the dental field has long utilized ceramics for tooth replacement. Additionally, the orthopedic field has found considerable use for ceramics in permanent joint and bone segment replacement and bone repair devices.

Ceramics exhibit a number of characteristics desirable for medical devices. Ceramics exhibit great strength and stiffness, resistance to corrosion and wear, and low density. Ceramic materials are generally biologically compatible and exhibit a high degree of stability following implantation. Ceramics can further be produced with voids and interstices that provide surfaces for bone ingrowth, thereby providing skeletal fixation for the permanent replacement of bones and joints.

Unfortunately, while generally exhibiting great strength, ceramic medical devices often exhibit poor fatigue resistance and are susceptible to fracture in use. This is due, at least in part, to thermal gradient effects, e.g. cracking and residual stress, which may develop during production of ceramic medical devices by conventional means.

Ceramic medical devices are generally made by forming raw ceramic materials into shapes that are roughly held together, known as “green bodies.” Green bodies are then heated by conventional means, e.g. atmospheric or pressure controlled furnaces, wherein the ceramic bodies are fused together into a solid mass. The fusion of the ceramic powder at a high temperature, wherein the body is consolidated into a desired shape, is called sintering.

A problem associated with conventional systems is that they heat by thermal transmission, with the internal regions of a green body being heated at a different rate than the external regions, resulting in said thermal gradient effects. Conventional systems are also inefficient and can require extended operational times in order to reduce residual stress and avoid cracking. The high temperatures and long heating times can also lead to undesired decomposition in the ceramic materials being sintered.

Accordingly, there is a continuing interest in developing ceramic medical devices that have reduced thermal gradient effects, and which are more rapidly and efficiently sintered in comparison to ceramic medical devices of the art.

### SUMMARY

A process for making a sintered ceramic medical device includes providing an unsintered ceramic composition, forming the unsintered ceramic composition into a green body that comprises unsintered ceramic, irradiating the green body with microwave radiation, and cooling the sintered ceramic medical device. The frequency of the radiation may be selected based on the excitation frequencies of the particular ceramic materials in the composition. The selected microwave radiation can be capable of volumetrically heating the unsintered ceramic to a temperature sufficient to consolidate the green body, thereby preparing the volumetrically sintered ceramic medical device.

Volumetrically sintered ceramic medical devices may be formed by various means including casting, compaction in a die under isostatic pressure, compaction onto the surface of a substrate, extrusion, immersion, spraying and injection molding. The ceramic itself may be sintered from ceramic powder or compositions comprising ceramic powder, for example, ceramic slurry. Ceramic powders may include powdered oxides such as alumina and/or zirconia, nitrides such as silicon nitride; stabilized ceramics such as magnesia-stabilized zirconia and yitria-stabilized zirconia; and doped ceramics such as silicon nitride with dopants such as yitria, magnesium oxide, strontium oxide, alumina, and combinations thereof. Solvents, if used to make ceramic slurry, may be polar or nonpolar. Ceramic compositions may also include reinforcing fillers, such as metal fibers, where the metal is preferably inert to ceramic at sintering temperatures and biocompatible. Such metal fibers include tantalum, gold, tungsten, and combinations thereof. Substrates used for forming composite ceramic medical devices can be metal, and may further be perforated if the medical application so requires.

Particular medical devices according to the disclosure include devices for bone and/or joint replacement. Such medical devices can comprise weight-bearing bone void filler or a replacement for a bone having an articulation surface, such as an acetabular shell, glenoid replacement, spinal implants for vertebral body replacements or patella replacement. In various embodiments, such replacement is a result of surgical procedure, degenerative disease, or trauma. In some embodiments, a ceramic medical device is polished to provide a suitable articulation surface, and can also have a separate section comprising of pores or perforations to promote bone ingrowth. The ceramic medical devices of the disclosure exhibit reduced residual stress and associated cracking, and are produced more efficiently in comparison to ceramic medical devices made by conventional processes.

Further areas of applicability will become apparent from the description provided herein. It should be understood that the description and specific examples are intended for purposes of illustration only and are not intended to limit the scope of the present disclosure.

### DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. The following definitions and non-limiting guidelines must be considered in reviewing the description of the technology set forth herein.

The headings (such as “Introduction” and “Summary,”) and subheadings used herein are intended only for general organization of topics within the disclosure of the invention, and are not intended to limit the disclosure of the technology or any aspect thereof. In particular, subject matter disclosed in the “Introduction” may include aspects within the scope of the present technology, and may not constitute a recitation of prior art. Subject matter disclosed in the “Summary” is not an exhaustive or complete disclosure of the entire scope of the technology or any embodiments thereof.

The citation of references herein and during prosecution of this application does not constitute an admission that those references are prior art or have any relevance to the patentability of the technology disclosed herein. Any discussion of the content of references is intended merely to provide a general summary of assertions made by the authors of the references, and does not constitute an admission as to the accuracy of the content of such references.

The description and specific examples, while indicating embodiments of the invention, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make, use and practice the devices and methods of this technology and, unless explicitly stated otherwise, are not intended to be a representation that given embodiments of this technology have, or have not, been made or tested.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

As used herein, the word “include,” and its variants, is intended to be non-limiting, such that recitation of items in a list is not to the exclusion of other like items that may also be useful in the materials, compositions, devices, and methods of this technology.

As used herein, the term “about,” when applied to the value for a parameter of a device or method of this technology, indicates that the calculation or the measurement of the value allows some slight imprecision without having a substantial effect on the chemical or physical attributes of the device or method. The terms “a” and “an” mean at least one. Also, all compositional percentages are by weight of the total composition, unless otherwise specified.

The present technology includes processes for making a sintered ceramic medical device, comprising

(a) providing a ceramic composition;

(b) forming the ceramic composition into a green body; and

(c) irradiating the green body with microwave radiation, said microwave radiation having a frequency capable of volumetrically heating the ceramic composition to a temperature sufficient to sinter the green body. In various embodiments, the irradiation and subsequent cooling is controlled so as to inhibit the formation of thermal gradient effects.

The devices made according to the disclosed processes may be used for the treatment of tissue defects in humans or other animal subjects. Specific materials to be used in the devices must, accordingly, be biomedically acceptable. As used herein, such a “biomedically acceptable” material is one that is suitable for use with humans and/or animals without undue adverse side effects (such as toxicity, irritation, and allergic response) commensurate with a reasonable benefit/risk ratio. As referred to herein, such “tissue defects” include any condition involving tissue which is inadequate for physiological or cosmetic purposes. Examples of such defects include those that are congenital, those that result from or are symptomatic of disease or trauma, and those that are consequent to surgical or other medical procedures. Examples of such defects include those resulting from osteoporosis, spinal fixation procedures, hip, knee, elbow and other joint replacement procedures, chronic wounds, and fractures. In various embodiments, such replacement is a result of surgical procedure, degenerative disease, or trauma.

In various embodiments, the present disclosure provides ceramic medical devices produced by a process wherein the ceramic is volumetrically sintered. As used herein, the term “volumetric” means uniform through the volume of the ceramic. Accordingly, the term “volumetrically sintered medical device” means that the ceramic medical device was heated uniformly through the volume of the medical device to a temperature at which the ceramic was uniformly sintered. Microwave technology offers a means of volumetrically consolidating ceramic medical devices and reducing thermal gradient effects. An advantage lies in an efficient use of energy to selectively excite and heat specific molecules within the material, rather than rely on thermal transmission from one zone to the other in the body of the ceramic, i.e. from the outside to the inside of a ceramic device. Thermal excitation can thus be efficiently utilized to volumetrically sinter ceramics and metal-ceramic composites. Higher heating rates may also be achieved, reducing the time necessary for sintering the ceramic.

The process, in the context of the present disclosure, comprises a step of providing an unsintered ceramic composition. The unsintered ceramic composition may comprise a dry, finely divided ceramic powder. The composition may comprise additional dry materials and additives. Alternatively, the composition can comprise a damp powder or a ceramic slurry made using either aqueous or organic liquid. Damp powder or slurry may further comprise additional materials and additives, for example a binder.

Suitable ceramic powders include structural ceramics, as opposed to ceramic powders that are resorbable, for example, hydroxyapatite and calcium phosphate. Suitable structural ceramic materials may be prepared from a variety of materials, including ceramics that are known for use in the art, including any one or more ceramic oxides or non-oxides, including carbides, borides, nitrides, and silicides. Particular oxides may include alumina and zirconia. Zirconia can be chemically “stabilized” in several different forms, including magnesia-stabilized zirconia and yttria-stabilized zirconia. Particular non-oxides may include silicon nitride and silicon carbide. Doped ceramics may also be used, such as yitria, magnesium oxide, strontium oxide, alumina, and combinations thereof. As a nonlimiting example, typical particle size distribution of ceramic powders may range from about 0.1 μm to about 200 μm in diameter, dependant upon the powder composition and morphology. The average particle size of ceramic powders for ceramic medical devices generally may be less than 10 μm in diameter, even more generally less than 5 μm in diameter, and most generally less than 1 μm in diameter.

A ceramic composition may comprise a ceramic slurry comprising a ceramic powder and a solvent. Ceramic slurries may be produced by means known in the art. For instance, a slurry may be produced by mixing a ceramic powder with a liquid solvent, whereby the ceramic particulates are suspended in the liquid. Suitable solvents can be comprised of one or more polar or non-polar liquids, including liquids such as water, aqueous solutions, acetone, alcohols, organic solvents, and halogenated solvents. Alcohols may include C₁-C₈ alcohols, such as ethyl alcohol, butyl alcohol, isopropyl alcohol, and the like. Organic solvents may include aromatic solvents, such as toluene and the like. Suitable halogenated solvents may include chlorinated solvents such as methylene chloride, tetrachloromethane, and the like.

A liquid solvent may be capable of vaporizing at ambient or non-sintering temperatures prior to consolidation or sintering, or at the temperatures reached during sintering of the ceramic medical devices of the disclosure. The polarity of the solvent can be chosen based on the solubility characteristics of other slurry materials. For example, a solvent may be chosen such that space fillers do not dissolve in the solvent.

Binders may also be included in the ceramic slurry of the disclosure. Binders can be used to increase the cohesiveness of a ceramic composition. Binders generally decompose into volatile and/or gaseous residues, or oxidize at or below the temperature at which sintering occurs. Suitable binders can include organic materials with a melting point of less than about 300° C. Suitable organic binders are generally hydrocarbon polymers that decompose at the high temperatures associated with the sintering process. Nonlimiting examples of suitable organic binders include waxes, for instance paraffin wax, polyethylene glycol, polyvinyl alcohol, carboxymethyl cellulose, and combinations thereof.

Binders may further comprise resins or polymers such as polyethylene, polypropylene, polyvinyl acetate, and polyvinyl butyral. Acrylic binders formed from alkyl acrylate and the alkyl methacrylate monomers, wherein the monomers have an alkyl group having from 1 to 8 carbon atoms, are also suitable. Nonlimiting examples of such (meth)acrylic monomers include methyl acrylate, ethyl acrylate, isopropyl acrylate, n-butyl acrylate, isobutyl acrylate, cyclohexyl acrylate, 2-ethylhexyl acrylate, methyl methacrylate, ethyl methacrylate, isopropyl methacrylate, n-butyl methacrylate, isobutyl methacrylate, cyclohexyl methacrylate, and 2-ethylhexyl methacrylate. Polymeric and resin binders generally have weight average molecular weight (Mw) between about 10,000 to 500,000, selected so that the aggregation force of the binder and the overall viscosity of the slurry is sufficient for forming a green body.

Ceramic slurry may further comprise a non-dissolving space filler. Examples of non-dissolving space filler include, but are not limited to, ammonium bicarbonate, polystyrene, and urea. Such space fillers are preferably non-dissolving, such that they do not dissolve in the solvent of the slurry. For example, ammonium bicarbonate would be chosen as the space filler if the solvent were a non-polar liquid, such as toluene. Polystyrene would generally be chosen as the non-dissolving space filler if the solvent comprises water or alcohol. In various embodiments, the material selected as the non-dissolving space filler dissociates or sublimes during the sintering process, resulting in a porous sintered ceramic.

It should be further understood that ceramic slurry can comprise additional additives. Nonlimiting examples of additional additives include molding adjuvants such as dispersion agents, antifoamers, for example 1-butanol, and antistatic agents.

The ceramic composition of the disclosure may further comprise reinforcing materials, for example metal filler. Preferably, the metal is inert to the ceramic, and is biocompatible. Suitable metals may include one or more of tantalum, gold, tungsten, cobalt, chromium, titanium, and alloys thereof. The metal filler can be present in various shapes such as randomly shaped particles, spherical powder, fibers, whiskers, rods, or random shapes. In general, the fillers should have an elongated shape, such as a fiber, for further strengthening and reinforcing of the ceramic. The aspect ratio of a metal filler particles may be such that the fibers are larger than a critical length L_{c}, defined as the minimum length at which the center of a fiber reaches the ultimate (tensile) strength s_{f}, when the matrix achieves the maximum shear strength tₘ, or L_{c}=s_{f}d/(2 tₘ). Since L_{c} is proportional to the diameter of the fiber d, effective strengthening may also be achieved with an aspect ratio of L/d>s_{f}/(2 tₘ).

Reinforcing materials may further be continuous in nature. For example, the metal filler of the disclosure may comprise a metal mesh or matrix, or continuous metal filaments or wires that provide reinforcement to the medical device. Reinforcing materials may further include non-metal materials, such as carbon or silica based fillers that do not decompose or dissociate at temperatures sufficient for sintering of ceramics.

The process further comprises a forming step. Ceramic medical devices are generally made by forming raw ceramic materials into shapes that are loosely held together. In the art, these loosely held together shapes are known as “green bodies.” Green bodies may be formed by various means including casting, compaction in a die under isostatic pressure, compaction on the surface of a substrate, extrusion, immersion, spraying, and injection molding.

In the case of casting, a ceramic slurry may be cast in a mold according any method known in the art. Casting of ceramics may be performed at room temperature. A green body may be cast and then sintered, wherein solvent and any binder and/or nondissolving filler is vaporized, oxidized, or otherwise dissociated, resulting in a sintered ceramic object. Alternatively, the slurry ceramic particles may first be suspended in a liquid and then cast and dried, or cast into a porous mold that removes the liquid, leaving a particulate compact in the mold for sintering.

Ceramics may also be formed by compacting a dry or slightly damp ceramic powder, with or without an organic binder, in a die. Compaction may be effected with an isostatic press. It should be understood that a wide array of pressures may be chosen based on such variables as the particular ceramic composition being compacted and the end-use of the particular medical device being formed. For example, pressures may generally range from about 15 psi to about 400,000 psi. Suitable pressures for compaction of green bodies according to the disclosure may be about 50,000 psi.

Forming may also comprise compaction of ceramic compositions onto substrates to produce ceramic composites following sintering. Substrates may include metal objects, such as metal domes for acetabular shells. Nonlimiting examples of metals suitable for composite medical devices may include tantalum, tungsten, cobalt, chromium, titanium, and combinations or alloys thereof.

The process of the disclosure further comprises irradiating a formed green body with microwave radiation emitted by a microwave generator. It should be understood that any microwave generator capable of producing the microwave frequencies of the disclosure may be suitable for use in the sintering process. The microwave equipment may comprise a magnetron and a resonant cavity connected by a waveguide. The power and frequency capable of being emitted by the generator may be adjustable.

Microwaves are electromagnetic waves in the frequency band from about 300 MHz to about 300 GHz. Industrial microwave processing is usually accomplished at the frequencies set aside for industrial use, i.e. 915 MHz, 2.45 GHz, 5.8 GHz, and 24.124 GHz. However, because ceramic materials are “transparent” to certain frequencies of microwave energy, and microwaves of particular frequencies can pass through ceramic without being absorbed, it should be understood that the radiation frequencies selected for the process of the disclosure are based on the particular excitation frequencies of ceramic materials in the composition. Furthermore, microwave power may also be adjusted to affect rate of heating and/or cooling of ceramics.

For example, ceramics are known dielectric materials that have a permittivity (ε) in the microwave regions from which a dielectric loss (ε″) or the related loss tangent δ (wherein tan δ equals the ratio of the dielectric loss to the relative permittivity ε′, or ε″/ε′) may be calculated. Such loss values indicate the proportion of microwave energy absorbed by the material and dissipated in the form of heat. It should be recognized that the dielectric loss of ceramic materials, having known dependencies on temperature and microwave frequency, may be determined and used to select frequencies that thermally excite the ceramic. It should also be recognized that because dielectric loss is temperature dependent, ceramic materials may be preheated by conventional or other means to critical temperatures, wherein the absorption of radiation is more effective for the ceramic material.

During the time that ceramic is exposed to penetrating microwave radiation, some energy is irreversibly lost through absorption by the ceramic material which in turn generates heat within the volume or bulk of the ceramic. This bulk heating raises the temperature of the ceramic material volumetrically, such that the interior portion of a green body heats at the same rate and to the same temperature as the exterior surface, especially when the surface does not significantly lose heat to cooler surroundings. This is the reverse of conventional heating, where heat from an external source is supplied to the exterior surface and diffuses toward the cooler interior regions.

Furthermore, the ceramics of the disclosure may be exposed to radiation having more than one frequency. For instance, where a ceramic composition comprises more than one ceramic or other material capable of coupling with microwave radiation to produce heat, radiation with more than one frequency may be used to excite multiple materials. Frequencies may be selected that interact not only with the ceramic composition, but also with any additional materials or substrates, such as in the case of composite ceramic medical devices having metal substrates. Furthermore, continuous adjustment of microwave frequency and power during heating may be performed based on changes in dielectric loss that may occur with change in temperature of the ceramic.

Without limiting the scope, function or utility of the present technology, in various embodiments, microwave heating provides several benefits, including rapid heating without overheating the surface, reduced surface degradation during drying, and removal of solvents and binders from the interior of the ceramic without cracking. Higher heating rates can also result in better densification. For example, in various embodiments, ceramic medical devices sintered according to the process of the disclosure have a density of greater than about 85% of theoretical density, and preferably greater than about 95%. In one embodiment, the density is greater than about 99% of theoretical density. Rapid microwave heating can also reduce the ultimate temperature necessary to achieve densification. Improved rapid heating to lower ultimate temperatures can lead to the production of denser ceramic materials with finer grain size.

The frequency or frequencies of microwave radiation selected may be capable of volumetrically heating the unsintered ceramic to a temperature sufficient to sinter the green body, thereby preparing a volumetrically sintered ceramic medical device. As referred to herein, “volumetric” heating refers to heating the ceramic body by means other than surface heating. Preferably the heating is substantially throughout the entire ceramic body. In various embodiments, prior to sintering, a green body may be preheated to a temperature sufficient to vaporize, burn, or otherwise dissociate any solvent and binder. As a nonlimiting example, preheating up to a temperature of approximately 700° C. may be conducted. Preheating may be performed, for example, to adjust the dielectric loss of the ceramic material and increase the absorption of microwave radiation, or to remove the binder from the ceramic composition. Removal of binders is generally known as “debinding.” Following any preheating or debinding step, the temperature may be raised further to a temperature sufficient for sintering.

Sintering temperatures vary widely and are primarily based on the nature of the ceramic materials selected for firing by irradiation. As a nonlimiting example, sintering may occur from about 700° C. to about 2000° C., although it should be understood that higher or lower temperatures may be necessitated by the particular materials comprising the unsintered ceramic composition.

It should be understood that the time required to increase the temperature of the ceramic will vary based on the ceramic material and frequency of radiation chosen, although the time required for heating by microwave irradiation is generally much lower than observed in conventional heating.

Microwave sintering according to the disclosure may be performed under a vacuum or an inert atmosphere to avoid reaction of the ceramic with atmospheric oxygen and/or nitrogen. An inert atmosphere generally may include one or more noble gases, for instance helium, neon, argon, krypton, xenon, or combinations thereof.

The microwave sintering of the disclosure may be performed with a susceptor bed having free flowing granules of a microwave susceptor material, and a minor amount of a refractory parting agent either dispersed in, or coated on, the susceptor material to prevent sintering of the susceptor material. Such a susceptor bed may surround the green body and can also be thermally excited at the microwave frequencies of the disclosed process, whereby the exterior surface of the green body may avoid significant temperature loss in comparison to the interior of the green body. Additionally, the susceptor bed may provide a means of preheating the green body to a temperature sufficient for microwave coupling to heat the green body, particularly when the susceptor material and/or parting agent are capable of coupling with microwave radiation below the critical temperature of the ceramic composition.

In various embodiments, the process of the disclosure further comprises a cooling step, wherein the formation of thermal gradient effects, such as residual stresses and/or cracks, is inhibited, and mechanical failure in the form of thermal shock is prevented. Thermal shock is the name given to cracking as a result of rapid temperature change. Thermal shock occurs when a thermal gradient causes different parts of the ceramic to expand differently. Cooling can be performed by a reduction in microwave irradiation power or a change in frequency to a frequency that the ceramic absorbs less effectively. The sintered ceramic may also be slowly cooled by introducing a flow of inert gases. Additional annealing steps may further be performed to reduce thermal gradient effects following the cooling step.

Medical devices comprising volumetrically sintered ceramic may be processed to modify characteristics such as shape or texture. Medical devices may be polished, for example with a diamond wheel, to provide a smooth surface suitable for use as an articulating surface in joint replacement. Such a suitable surface may have a roughness of less than 1 μm, and is preferably less than 100 nanometers, more preferably less than 50 nanometers. In one embodiment, the roughness is about 20 nanometers. Further finishing and machining of the ceramic medical device may also be required to adjust the device shape prior to the end-use of the device. It should also be understood that, in some instances, forming may only be performed on ceramics after sintering, for example by machining into a suitable device shape.

The ceramic medial devices of the disclosure may be solid, especially for implants used in load-bearing applications and/or applications in which complete bone ingrowth is not possible. Alternatively, or in combination, the ceramic medical devices may be porous for simulation of cancellous or spongy bone, allowing improved interconnectivity of the implant with existing bone structure. It should be understood that adequate pore size may vary based on the application of the medical device, and pore size may be selectively adjusted according to the process of the disclosure. As nonlimiting examples, pore size for mineralization may be larger than 150 μm, and adequate size for interconnection may be approximately 75 μm. Also, a pore diameter of 200 μm corresponds to the average diameter of an osteon in human bone, while a pore diameter of 500 μm corresponds to remodeled cancellous bone. In various embodiments, pores range in size from about 50 μm to about 600 μm in diameter. Open cell structures can be fabricated to virtually any desired porosity and pore size, and can thus be matched perfectly with the surrounding natural bone in order to provide an optimal matrix for ingrowth and mineralization. Furthermore, medical devices having metal substrates according to the disclosure may have perforations for promotion of bone ingrowth. Perforations may range in size from about 50 μm to about 600 μm in diameter.

Volumetrically sintered ceramic medical devices according to the disclosure include osseous implants such as weight-bearing bone void filler. Nonlimiting examples of void filler are weight-bearing filler for segmental defects or spinal grafts. Ceramic medical devices further include orthopedic implants and replacements, for example, replacements for bones with articulation surfaces, such as acetabular cups, femoral components for the knee, glenoid replacements, or patella replacements.

Volumetric sintering results in ceramics with reduced thermal gradient effects in comparison to conventional ceramics. Medical devices comprising volumetrically sintered ceramic exhibit little or no cracking and residual stress. The efficient use of microwave energy to selectively excite and heat specific molecules, volumetrically sintering ceramics and metal-ceramic composites, addresses the poor fatigue resistance and susceptibility to fracture observed with conventional ceramic medical devices. Higher heating rates achievable through use of volumetric microwave sintering further reduce the production times for ceramic medical devices.

The devices and methods of this technology are further illustrated by the following non-limiting examples.

### EXAMPLE 1

Alumina particles of size range less than 1 micron are placed in a flexible (rubber) mold and sealed with a rubber stopper with an opening for evacuation. The rubber mold is generally shaped similar to the final desired shape. For example, an acetabular shell may be compacted in a mold that is a hemisphere. The dimensions of the mold are substantially larger than the final dimensions of the finished sintered material and compaction results in decreased dimensions and subsequent sintering will result in further shrinkage. A vacuum pump with filter is connected to the rubber mold and the air inside the mold is pumped out. This operation allows for the removal of entrapped air between the alumna particles. If not incorporated in the operation, the compacted powder may crack due to the expansion of air after the compaction process. The mold is sealed and disconnected from the vacuum pump. The sealed mold is placed in a cold isostatic press (CIP) machine and pressurized to about 50000 psi for about 1 to 5 minutes. The isostatic pressure allows for uniform compaction. The compacted alumina (green material) is easily removed from the mold (due to the shrinkage in dimensions) and may be machined if required. Depending on the shape, a pre-sintering operation at about 900° C. in a conventional oven may be needed to allow for increased green strength, a desirable feature during green machining. The green machined part is placed in a 1500 Watt microwave furnace with a frequency of either 915 MHz or 2.45 GHz. The power of the microwave furnace is gradually increased by about 25 Watts every 10 to 15 minutes. The ramp-up of the power of the microwave is empirically determined for every shape and size such that the heating rate is at a preferred rate of less than 2° C. a minute. This allows for gradual but volumetric heating of the parts to temperatures in excess of a 1000° C. (preferably 1350° C.). Depending on the size of the part, the heating time is generally between 30 to 180 minutes. After the soak time at these sintering temperatures, the power of the microwave furnace is gradually decreased, for example by 25 Watts, every 10 to 15 minutes and the parts are allowed to cool at a preferred rate of less than 2° C. per minute. The part is removed from the furnace when the part temperature is such that the part can be touched by bare hands. After the sintering process, the articulating surface (if any) is polished with successively decreasing particle size of diamond polishing compound to a roughness less than 50 nanometers.

### EXAMPLE 2

Tantalum powder of average particle size of about 10 microns but all particles less than 15 microns is placed in a rubber mold which has its inside cavity walls precoated with some fluid that allows the tantalum to stick on the mold wall. This fluid could be d-limonine or mold release compound or any other organic which burns off at a low temperature (less than 400° C.). This coating is desired to achieve a coating of the metal powder on the inside walls of the mold cavity. Excess powder is drained out of the mold cavity. Alumina powder of particle size less than 1 micron is poured into the mold lined with metal powder. This is then placed in a vibratory unit that vibrates the unit to allow the powder to settle into the crevices and pores of the metal layer and further build-up as single phase alumina with no metal. This composite structure, where one zone has metal-ceramic and the other is only ceramic is placed in a microwave furnace sintered as described in paragraph [0054].

### EXAMPLE 3

A very thin layer of alumina particles of size range less than 1 micron is placed in a flexible (rubber) hemispherical mold. A hemispherical tantalum wire cage is placed inside this pre-coated rubber mold such that the metal cage is seated in the bed of alumina. Further, more alumina is added to this construct so as to build up a thick layer, possibly submerging the metal cage. This composite structure, where one zone has metal-ceramic and the other is only ceramic, is cold, isostatically pressed, and then placed in a microwave furnace sintered as described in paragraph [0054].

The examples and other embodiments described herein are exemplary and not intended to be limiting in describing the full scope of compositions and methods of this invention. Equivalent changes, modifications and variations of specific embodiments, materials, compositions and methods may be made with substantially similar results. For example, ceramic powder such as magnesium oxide stabilized zirconia can be used instead of alumina, and other high temperature metals may be used instead of tantalum. Further, the examples above describe fabrication of acetabular shells, however the concept may be used for making knee and other components.

What is claimed is:

1. A process for making a sintered ceramic medical device, comprising:
providing a ceramic composition;
forming the ceramic composition into a green body; and
sintering the green body by irradiating with microwave radiation, said microwave radiation having a frequency capable of heating the ceramic composition to a temperature sufficient to sinter the green body.

2. A process according to claim 1, further comprising cooling the sintered body, whereby said sintering and cooling are controlled so as to inhibit the formation of thermal gradient effects.

3. A process according to claim 1, wherein the heating of the green body is volumetric.

4. A process according to claim 1, comprising preheating the green body to a critical temperature prior to the step of irradiating the green body, wherein the critical temperature is a temperature at which the dielectric loss of the ceramic composition is sufficient for the composition to be thermally excited upon irradiation with microwave radiation.

5. A process according to claim 1, wherein the ceramic composition comprises a ceramic powder selected from the group consisting of oxides, carbides, borides, nitrides, silicides, and mixtures thereof.

6. A process according to claim 5, wherein the ceramic powder is selected from the group consisting of alumina, zirconia, magnesia-stabilized zirconia, yttria-stabilized zirconia, silicon nitride, silicon carbide, and mixtures thereof.

7. A process according to claim 1, wherein the ceramic composition is sintered to greater than 95% theoretical density.

8. A process according to claim 1, wherein the ceramic composition comprises a slurry comprising:
a) a ceramic powder; and
b) a solvent.

9. A process according to claim 8, wherein the solvent is selected from the group consisting of: water, acetone, alcohols, organic solvent, halogenated solvent, and mixtures thereof.

10. A process according to claim 8, wherein the ceramic slurry further comprises a binder.

11. A process according to claim 8, wherein the ceramic slurry further comprises a non-dissolving space filler wherein the non-dissolving space filler does not dissolve in the solvent of the ceramic slurry.

12. A process according to claim 11, wherein the sintered ceramic medical device is porous.

12. A process according to claim 8, wherein the ceramic slurry further comprises a non-dissolving space filler wherein the non-dissolving space filler does not dissolve in the solvent of the ceramic slurry.

13. A process according to claim 5, wherein the forming of the device shape comprises compacting the ceramic powder in an isostatic press.

14. A process according to claim 13, wherein the forming of the device shape comprises compacting the ceramic composition onto a metallic substrate.

15. A process of claim 1, wherein the ceramic composition further comprises metal filler.

16. A process according to claim 1, wherein the step of heating the ceramic composition comprises heating the ceramic composition in a vacuum or under inert gas atmosphere.

17. A ceramic medical device comprising a volumetrically sintered ceramic.

18. A ceramic medical device according to claim 17, wherein the ceramic has a theoretical density greater than 95 percent.

19. A ceramic medical device according to claim 17, wherein the ceramic is porous.

20. A ceramic medical device according to claim 17, wherein the ceramic is nonporous.

21. A ceramic medical device according to claim 17, further comprising a metallic substrate.

22. A ceramic medical device according to claim 17, wherein the medical device comprises a porous, weight-bearing bone void filler.

23. A ceramic medical device according to claim 17, wherein the device comprises a bone replacement having an articulation surface.
